(19) United States
(12) Patent Application Publication
(10) Pub. No.: US 2026/0347746 A1
(43) Pub. Date: Nov. 5, 2026

(54) INTEGRATED AUTONOMOUS CELL CULTURE PLATFORM WITH ROBOTIC HANDLING AND EMBEDDED AI AGENT FOR OPERATION

(71) Applicant: MitoAI, Inc., Boston, MA (US)

(72) Inventors: Bingda Li, Pittsburgh, PA (US); Xinyu Wang, Pittsburgh, PA (US)

(73) Assignee: MitoAI, Inc., Boston, MA (US)

(21) Appl. No.: 19/559,827

(22) Filed: Mar. 6, 2026

Related U.S. Application Data
(63) Continuation-in-part of application No. 19/074,039, filed on Mar. 7, 2025.
(60) Provisional application No. 63/946,524, filed on Dec. 22, 2025.

(51) Int. Cl. C12M 1/36; B25J 9/16; C12M 1/00

(52) U.S. Cl. CPC C12M 41/48; B25J 9/1612; B25J 9/1697; C12M 41/14

(57) ABSTRACT

A cell incubation system comprising a dark incubation chamber configured to maintain monitored temperature, humidity, and filtered air, the chamber comprising a multi-level shelving assembly having a vertical load-bearing central pillar defining a rotational axis and a plurality of shelf racks spaced along the pillar at different elevations, each shelf rack including a mounting hub configured to rotatably engage the pillar. The system further comprises a cell image monitor system configured for real-time microscopic imaging, comprising a movable objective lens module, fiber-optic transmission system, and remote imaging unit. The system further comprises an operation hood with at least one cell vessel operation station and a 3-axis robotic gantry system coupled with at least one liquid handling device, and a robotic transportation system with at least one robotic arm configured to transport cell vessels between the incubation chamber and operation hood.

### CROSS-REFERENCE TO RELATED PATENT APPLICATIONS

This application claims priority to or is a continuation-in-part of or relates to U.S. Application No. 19/074039, titled “AI Manipulated Automated Cell Culturing System and Method’, filed Mar. 7, 2025, which is hereby incorporated by reference in its entirety. This application also claims priority to U.S. Provisional Application No. 63/946,524 filed Dec. 22, 2025, titled “AUTOMATED CELL CULTURE SYSTEM”, which claims priority of US Application No. 19/074039, which is hereby incorporated by reference in its entirety.

### STATEMENT OF GOVERNMENT INTEREST

Not Applicable.

### JOINT RESEARCH AGREEMENT

Not Applicable.

### FIELD OF INVENTION

The present disclosure relates generally to cell culture systems, and more particularly to an automated cell incubation system integrating robotic cell vessel handling, real-time microscopic imaging, and AI-assisted culture monitoring within a unified platform.

### BACKGROUND

Cell culture operations form a foundational component of biological research, pharmaceutical development, and therapeutic manufacturing. These operations involve maintaining living cells, tissues, or organoids in controlled environments that provide appropriate temperature, humidity, gas composition, and nutrient supply to support cellular growth and function.

Conventional cell culture workflows typically rely on incubators that maintain environmental conditions suitable for cell growth. Laboratory personnel perform routine tasks such as media exchange, cell passage, and microscopic observation by manually retrieving cell vessels from incubators, transporting them to separate workstations, and performing operations under biosafety cabinets before returning the vessels to the incubator. This workflow pattern has remained largely unchanged for decades and continues to be the standard approach in many research and production settings.

The manual handling inherent in conventional cell culture workflows presents several challenges. Each time an incubator door is opened, the internal environment experiences fluctuations in temperature, humidity, and gas composition. Cell vessels removed from incubators are exposed to ambient laboratory conditions during transport and manipulation. These environmental variations can affect cell behavior and culture outcomes. Additionally, the transfer of cell vessels between different workstations creates opportunities for contamination from airborne particles, surface contact, or operator handling.

As cell culture operations scale beyond small numbers of vessels, the labor demands of manual workflows increase proportionally. Personnel must dedicate substantial time to repetitive tasks such as media changes and visual inspections. The scheduling of these tasks across large numbers of vessels becomes increasingly complex, and the consistency of technique may vary between different operators or across different time points.

Microscopic observation of cell cultures in conventional workflows typically occurs at discrete intervals when personnel retrieve vessels for inspection. Between these scheduled observations, changes in cell morphology, growth patterns, or contamination events may progress undetected. The frequency of observation is constrained by the practical limitations of manual handling and the competing demands on laboratory personnel time.

Various approaches to automating portions of cell culture workflows have been developed. Microplate-based systems offer automated liquid handling and imaging capabilities for small-volume, high-throughput applications. At larger scales, bioreactor systems provide automated control of culture conditions for industrial production volumes. However, a gap exists between these approaches for operations that require handling of diverse vessel types at intermediate scales, where the flexibility of conventional vessel-based culture is desired but manual handling limitations constrain throughput and consistency.

The integration of robotic handling, environmental control, and imaging capabilities within cell culture systems represents an area of ongoing development. Such integration may address challenges associated with environmental stability, contamination risk, labor requirements, and monitoring frequency in cell culture operations. There is a demand for a cell incubation system that integrates automated cell vessel handling, real-time microscopic imaging, and AI-assisted culture monitoring within a unified platform, thereby eliminating the requirement for repeated manual intervention and enabling large-volume cell culture operations with improved consistency and efficiency

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a cell incubation system is provided. The cell incubation system includes a dark incubation chamber configured to maintain monitored temperature and humidity and filtered air. The incubation chamber comprises a multi-level shelving assembly having a vertical load-bearing central pillar defining a rotational axis and a plurality of shelf racks spaced along the pillar at different elevations. Each shelf rack includes a mounting hub configured to rotatably engage the pillar such that the shelf rack is rotatable about the axis between angular positions. The cell incubation system further includes a cell image monitor system configured to allow real-time microscopic imaging inside the cell incubation system. The cell image monitor system comprises a movable objective lens module, a fiber-optic transmission system, and a remote imaging unit. The cell incubation system further includes an operation hood comprising at least one cell vessel operation station and a robotic gantry system. The robotic gantry system is a 3-axis mechanical motion platform coupled with at least one liquid handling device. The cell incubation system further includes a robotic cell vessel transportation system having at least one robotic arm with a gripper configured to pick up and transport cell vessels between the incubation chamber and the operation hood.

According to other aspects of the present disclosure, the cell incubation system may include one or more of the following features. The operation hood may be adjacent to the dark incubation chamber with a shared window configured for cell culture vessels to be mechanically transported between the incubation chamber and the operation hood. The shelf racks may rotate independently. The multi-level shelving assembly may further comprise a coupling element configured to synchronize rotation of the plurality of shelf racks. The multi-level shelving assembly may further comprise at least one of rails, lips, dividers, and an indexing or locking mechanism configured to hold selected angular positions.

According to other aspects of the present disclosure, the movable objective lens module may comprise a small lightweight lens assembly positioned at a sample site and mounted on a mechanism that provides motions such as a linear actuator, a motorized slider, and a pivot joint. The mechanism may be electrically actuated, magnetically driven, servo-controlled, or manually adjusted through micro-mechanical linkages. The fiber-optic transmission system may comprise optical fibers with single-mode or multi-mode configurations that collect and channel light captured by the movable objective lens module. The remote imaging unit may comprise a CCD/CMOS sensor, a display module, and a computer interface configured to receive transmitted light and reconstruct it into a magnified image. The remote imaging unit may be configured to perform digital enhancement, focusing algorithms, or spectroscopic analysis.

According to other aspects of the present disclosure, the cell vessel operation station may comprise a base anchor on a floor of the operation hood, a heating plate surrounded with a peripheral guard wall to form a space for a cell culture vessel, at least one mechanical spring positioned between the cell culture vessel and the guard wall to facilitate a snug fit of the cell vessel within the space, and a wrist assembly and a rotary mechanism connecting the heating plate with the base anchor to control movement of the cell vessel for rotational movement or stationary positioning on a tilted position. The cell vessel operation station may further comprise a motorized cap opening unit having a motor, torque control, and an adaptive gripping head configured to automatically grip and twist open or close caps or lids of cell culture vessels. The motorized cap opening unit may further comprise a sensor configured to adjust gripping pressure and prevent slippage or over-tightening.

According to other aspects of the present disclosure, the robotic gantry system may comprise an X-axis configured for horizontal axis movement, a Y-axis mounted on the X-axis configured to move perpendicular across a workspace, and a Z-axis mounted on the Y-axis configured to move vertically to pick, place, approach, retract, or apply tools. The robotic gantry system may further comprise mounting points for rails, motors, and cable tracks, a bearing system comprising linear guide rails with recirculating ball carriages, and a drive mechanism for precision positioning such as belt drive, ball-screw drive, rack-and-pinion, linear motors, or other suitable drive mechanisms

The robotic gantry system may further comprise servo motors with absolute or incremental encoders for closed-loop control. The Z-axis may comprise a high-resolution ball-screw hinge, an integrated mechanical brake, and a lightweight carriage. The robotic gantry system may carry tools such as mechanical grippers, pneumatic grippers, vacuum grippers, cameras for inspection, dispensing heads, or other tools as suitable for the cell culture operation. The robotic gantry system may comprise a cable system having power cables, encoder lines, pneumatic tubing, and sensing wiring, and a liquid tubing system connected to a liquid storage system. The robotic gantry system may be connected to a control system comprising at least one industrial motion controller and one PC-based controller connected to an AI-command center. The liquid handling device may be an electronic pipette or an automatic device suitable for exchanging liquid for a cell culture unit. The robotic gantry system may have multiple Z-axes configured to carry synchronized liquid dispensing for more than one cell vessel.

According to other aspects of the present disclosure, the cell incubation system may further comprise at least one programmable mechanical manipulator having 4-7 degrees of freedom implemented within the operation hood and configured to operate in synchronization with the robotic gantry system. The programmable mechanical manipulator may comprise a base joint, multiple articulated segments connected by joints, rotary or linear actuators, a wrist assembly, and an end-effector.

The cell incubation system may further comprise a temperature and humidity control system configured to supply conditioned air flow for the incubation chamber and the operation hood. The cell incubation system may further comprise a waste collection and storage system having a solid waste container and a liquid waste container.

According to other aspects of the present disclosure, the cell incubation system may further comprise a liquid storage and supply system comprising liquid storage units located within a refrigerator, at least one liquid reservoir located within the operation hood, a liquid heating and cooling system, and a pressurized liquid tubing system connecting the liquid storage units and the liquid reservoir. The liquid reservoir may have a container positioned to be accessible for a pipette within the operation hood and connected to the pressurized liquid tubing system.

According to other aspects of the present disclosure, the robotic cell vessel transportation system may be configured to move cell vessels to designated locations such as a receiving plate within the incubation chamber, a microscopy observation station, and the cell vessel operation station. In some aspect, the cell vessel operation station may be a mechanical manipulator, such as a robotic arm with gripping function.

The foregoing general description of the illustrative embodiments and the following detailed description thereof are merely exemplary aspects of the teachings of this disclosure and are not restrictive.

### BRIEF DESCRIPTION OF FIGURES

### Brief Description of the Several Views of the Drawing

Non-limiting and non-exhaustive examples are described with reference to the following figures.

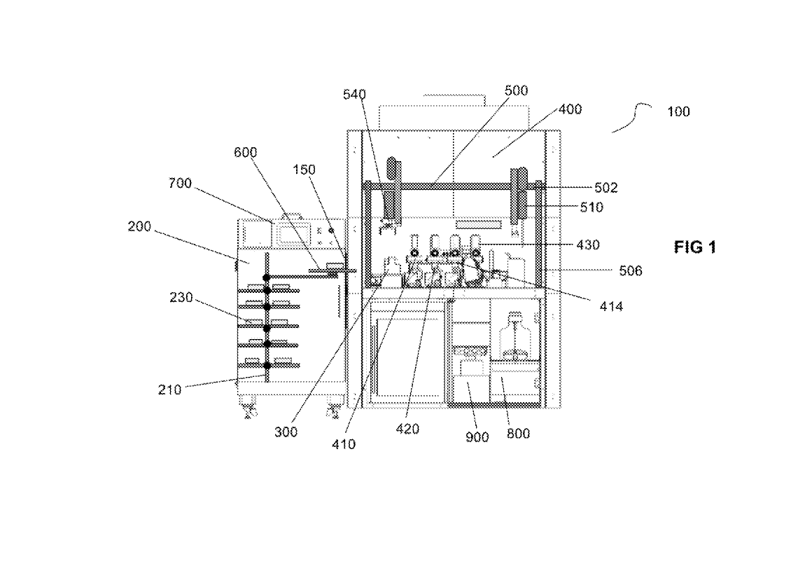

FIG. 1 is a front view of one of the embodiments of an overall view of an automated cell incubation system, according to aspects of the present disclosure.

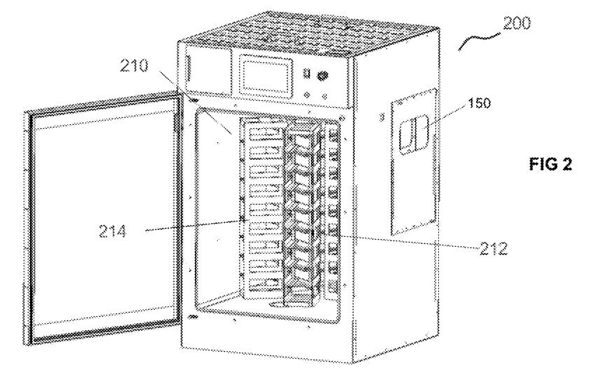

FIG. 2 is an isometric view of one of the embodiments of an incubation chamber with a multi-level shelving assembly, according to aspects of the present disclosure.

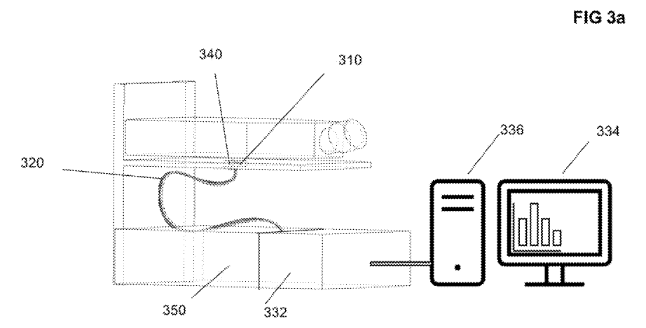

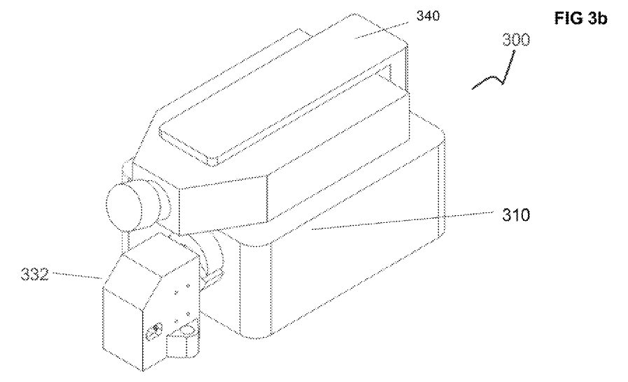

FIG. 3a is an isometric view of one of the embodiments of a cell image monitor system, according to aspects of the present disclosure.

FIG. 3b is an isometric partial view of a cell image monitor system with top light source, according to aspects of the present disclosure.

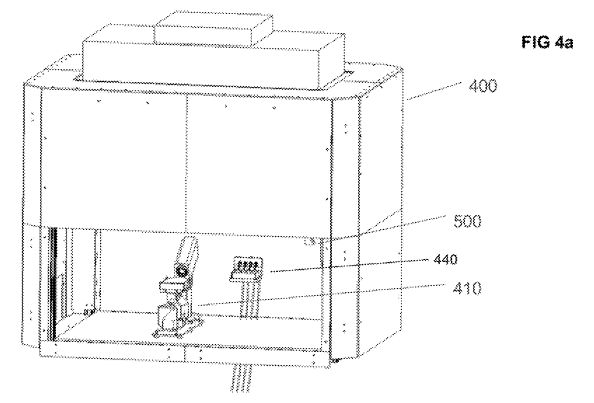

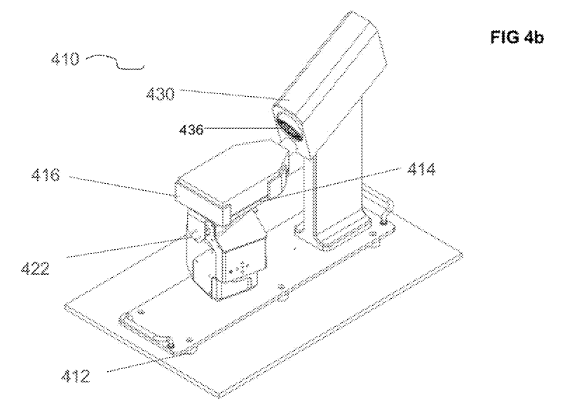

FIG. 4a is an isometric view of one of the embodiments of a cell vessel operation station within the operation hood, according to aspects of the present disclosure.

FIG. 4b is an illustration of one of the embodiments of a cell vessel operation station including the cap opener.

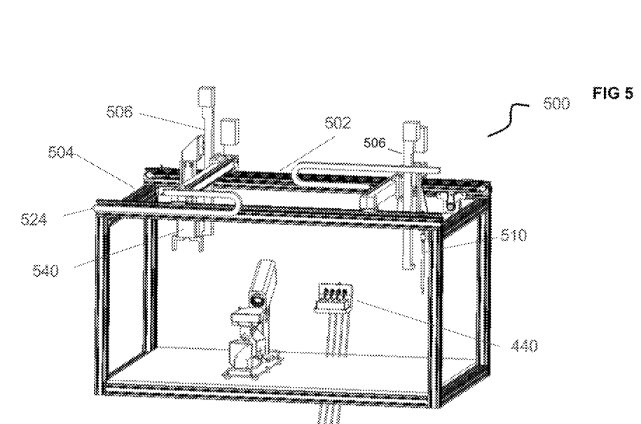

FIG. 5 is an isometric partial view the embodiments of the robotic gantry system, according to aspects of the present disclosure.

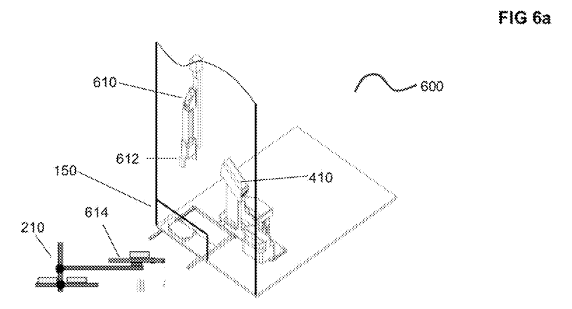

FIG. 6a is an isometric partial view of one of the embodiments of the robotic cell vessel transportation system, according to aspects of the present disclosure.

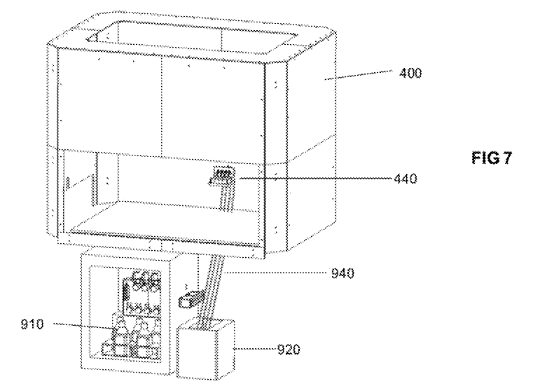

FIG. 7 illustrates a liquid storage and supply system in an isometric view, according to aspects of the present disclosure.

### DETAILED DESCRIPTION OF INVENTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A cell incubation system according to the present disclosure comprises a unified platform that integrates automated cell vessel handling, real-time microscopic imaging, and AI-assisted culture monitoring. The cell incubation system may address limitations associated with conventional cell culture workflows that rely on manual handling for routine tasks such as media exchange, cell passage, and microscopic observation. The cell incubation system may be adapted for AI-assisted culture monitoring and protocol generation. The AI-assisted culture monitoring may enable intelligent analysis of cell growth patterns observed through the cell image monitor system. The AI-assisted protocol generation may enable automated decision-making for routine culture operations such as media exchange, cell passage, and enzyme digestion. In some cases, the AI-assisted culture monitoring and protocol generation may reduce operator variability and ensure consistent execution of culture protocols across large numbers of cell vessels.

Referring now to FIG. 1, a system view for an automated cell incubation system 100 according to an embodiment of the present disclosure is illustrated. The cell incubation system 100 integrates automated cell vessel handling, real-time microscopic imaging, and AI-assisted culture monitoring within a unified platform. The system 100 comprises a dark incubation chamber 200 with monitored temperature and humidity and filtered air, a cell image monitor system 300 allowing real-time microscopic imaging, an operation hood 400 with a robotic gantry system 500 and liquid handling capabilities, and a robotic cell vessel transportation system 600 for automated movement of cell vessels between the incubation chamber 200 and operation stations. The system 100 further includes a temperature and humidity control system 700, a waste collection and storage system 800, and a liquid storage and supply system 900. The operation hood 400 is adjacent to the dark incubation chamber 200 with a shared window 150 that allows cell culture vessels to be mechanically transported between the incubation chamber 200 and the operation hood 400. The cell culture vessels may be transported between the two zones by delivering and receiving the cell culture vessels with a robotic arm from the operation hood 400 and a receiving plate from the incubation chamber 200.

The shared window may be configured to maintain environmental isolation between the dark incubation chamber and the operation hood when cell vessels are not being transported. In some cases, the shared window may comprise a door, gate, or other closure mechanism that opens to allow passage of cell vessels and closes to maintain separation between the two zones. The shared window may be sized and positioned to accommodate cell vessels of various sizes and configurations used in cell culture operations.

The integration of the dark incubation chamber, the cell image monitor system, the operation hood with the robotic gantry system, and the robotic cell vessel transportation system within the unified platform may eliminate the requirement for repeated manual intervention that characterizes traditional cell culture workflows. The cell incubation system may be adapted for large-volume cell, tissue, and organoid culture operations. In some cases, the cell incubation system may reduce contamination risk by minimizing the frequency with which cell vessels are exposed to external environments.

Incubation Chamber Referring now to FIG. 2, the dark incubation chamber 200 according to an embodiment of the present disclosure is illustrated. The dark incubation chamber 200 provides a controlled environment with monitored temperature and humidity and filtered air for cell culture operations. The incubation chamber 200 comprises a multi-level shelving assembly 210 having a vertical load-bearing central pillar 212 defining a rotational axis and a plurality of shelf racks 214 spaced along the pillar 212 at different elevations. Each shelf rack 214 includes a mounting hub 216 configured to rotatably engage the pillar 212 such that the shelf rack 214 is rotatable about the axis between angular positions. The rotatable configuration of the shelf racks may enable the robotic cell vessel transportation system to access cell vessels at different angular positions without requiring manual intervention. The multi-level arrangement of the shelf racks at different elevations may enable storage of large numbers of cell vessels within the dark incubation chamber. In some implementations, the shelf rack 214 rotates independently; in others, a coupling element 218 synchronizes rotation of multiple shelf racks 214. The coupling element 218 may be a shaft, rod, gear train, belt, chain, or other mechanical linkage extending between shelf racks at different elevations along the vertical load-bearing central pillar. Synchronized rotation provided by the coupling element may allow multiple shelf racks to be repositioned simultaneously to a common angular position. The shelving assembly 210 may further include features for stops, detents, or lock retention features such as rails 220, lips 222, dividers 224, and optional indexing or locking mechanisms 226 to hold selected angular positions. The incubation chamber 200 is configured to hold a plurality of cell vessels 230, such as flasks, Petri dishes, multiwell plates, multi-layer flasks, shaking flasks, or tissue culture chambers in various shapes that are suitable for a controlled, nutrient-rich environment that supports the growth, observation, and experimentation of living cells or tissue.

Cell Image Monitor System Referring now to FIG. 3a, the cell image monitor system 300 according to an embodiment of the present disclosure is illustrated. The cell image monitor system 300 allows real-time microscopic imaging inside the cell incubation system 100, comprising at least one movable objective lens module 310, a light supply 340, a fiber-optic transmission system 320, and a remote imaging unit 330. The movable objective lens module 310 includes a small lightweight lens assembly 312 positioned at a sample site. The sample site may be located within the incubation chamber or the operation chamber, or both. The objective lens module 310 is mounted on a mechanism 314 that provides motions, such as a linear actuator, motorized slider, or pivot joint. The motorization can be achieved by electrically actuated, magnetically driven, servo-controlled, or manually adjusted mechanisms through micro-mechanical linkages. The movable objective lens 312 allows focusing, scanning across a surface, or adjusting viewing angles. The fiber-optic transmission system 320 has optical fibers 322 with single-mode or multi-mode configurations that collect and channel the light captured by the lens 312 and light supply generated by a light source 350. The fiber 322 transmits the image data to a remote camera, photodetector, or eyepiece. The remote imaging unit 330 further comprises a CCD/CMOS sensor 332, a display module 334, and a computer interface 336. The remote imaging unit 330 receives the transmitted light and reconstructs it into a magnified image. The remote imaging unit 330 can include digital enhancement, focusing algorithms, or spectroscopic analysis capabilities. In some cases, the remote imaging unit 330 may convert optical signals received from the fiber-optic transmission system into electrical signals representing image data. The CMOS sensors may provide lower power consumption and faster readout speeds compared to CCD sensors. In some cases, the remote imaging unit may comprise both a CCD sensor and a CMOS sensor for different imaging applications.

FIG. 3b illustrate one of the other embodiments of the cell image monitor system where a light supply 340 is positioned from a different direction of the objective lens 310’s viewpoint, such as from the top of the cell sample. A fiber-optic transmission system connects the signals collected by the objective lens and the CCD/CMOS sensor 332. The CCD/CMOS sensor can be further connected to a display module and the remote imaging unit for further image processing and analysis.

In some cases, the cell image monitor system is configured to have fluorescence microscopy with necessary light sources, image filters, and optical mirror to reflect and transmit fluorescence light up toward the image detector such as the CCD/CMOS sensors in the remote imaging unit. In some other cases, the fluorescence microscopy system is a confocal system with image detectors such as PMTs and GaAsP photomultipliers.

The cell image monitor system may enable continuous observation of cell cultures without disturbing the controlled environment of the dark incubation chamber. The cell image monitor system may allow for early detection of morphological changes, contamination events, or other developments in cell cultures that might otherwise go unnoticed between scheduled manual inspections in conventional cell culture workflows.

The remote imaging unit may comprise a display module configured to present magnified images reconstructed from transmitted light received from the fiber-optic transmission system. The display module may enable visual observation of cell cultures by an operator. The display module may present real-time images of cell cultures captured by the cell image monitor system. The remote imaging unit may comprise a computer interface configured to transmit image data to external computing systems. The computer interface may enable storage, processing, and analysis of images captured by the cell image monitor system. The computer interface may provide image data to the AI-assisted culture monitoring system for intelligent analysis of cell growth patterns such as cell count, cell growth cycle analysis, cell apoptosis analysis, or any live cell imaging analysis based on the images captured by the system.

In one of the embodiments, as show in FIG. 4a, a partial overview of the operation hood 400 and the robotic gantry system 500, according to an embodiment of the present disclosure. The operation hood 400 comprises at least one cell vessel operation station 410 and the robotic gantry system 500.

Cell Vessel Operation Station Referring now to FIG. 4b, the cell vessel operation station 410 according to an embodiment of the present disclosure is illustrated. The cell vessel operation station 410 may be attached or removed with a base anchor 412 on the floor of the operation hood 400. The removable configuration of the cell vessel operation station may allow the cell vessel operation station to be detached from the base anchor for maintenance, cleaning, or replacement. In some cases, the base anchor may comprise mounting features such as threaded holes, slots, or quick-release mechanisms that facilitate attachment and removal of the cell vessel operation station.

The cell vessel operation station 410 further comprises a heating plate 414 surrounded with a peripheral guard wall 416. The space formed by the heating plate and the peripheral guard wall may accommodate cell culture vessels in various sizes. The heating plate may be configured to maintain cell culture vessels at controlled temperatures during cell culture operations such as media exchange or enzyme digestion. The heating plate may comprise heating elements and temperature sensors for regulating the temperature of cell culture vessels positioned on the heating plate. The peripheral guard wall may extend upward from a perimeter of the heating plate to define a bounded space above the heating plate. The peripheral guard wall may prevent lateral displacement of cell culture vessels from the heating plate during cell culture operation.

In some embodiments, optionally, a mechanical spring 418 that may be extended or pressed positioned between the cell culture vessel and the guard wall 416 to facilitate a snug fit of the cell vessel within the space when the cell vessel is placed on the heating plate 414 for operation. In some cases, multiple mechanical springs may be positioned at different locations around the peripheral guard wall to provide balanced securing forces on the cell culture vessel from multiple directions.

The heating plate 414 is connected with the base anchor 412 by a wrist assembly 420 and a rotary mechanism 422 to control movement of the cell vessel for rotational movement or stationary positioning on a tilted position as appropriate in routine cell culture operations such as media change or enzyme digestion. In some cases, tilted positioning of cell culture vessels may facilitate liquid handling procedures such as aspirating media from a corner of a cell culture vessel or distributing reagents across a cell culture surface.

In one embodiment, the rotary mechanism controls the movement without wrist assembly, with two independent joints for 2 degrees of freedom by using two planar 2-link arms, one rotational plus one linear joint, or two linear joints. The two independent joints enable movement in a 2D plane, a circular area, or a simple horizontal and vertical direction.

The wrist assembly and the rotary mechanism may be actuated by motors, servos, or other actuators that receive control signals from a control system of the cell incubation system. In some cases, the wrist assembly and the rotary mechanism may hold the heating plate in a stationary position at a selected tilted orientation during cell culture operations that require the cell culture vessel to remain at a fixed angle. The control of movement provided by the wrist assembly and the rotary mechanism may enable automated execution of routine cell culture operations such as media change or enzyme digestion that benefit from controlled positioning of cell culture vessels.

The cell vessel operation station 410 further comprises a motorized cap opening unit 430. The cap opening unit 430 has a small motor 432 to automatically grip and twist open or close the caps or lids of the cell culture vessel or other containers appearing within the operation hood 400. The cap opening unit 430 comprises a motor 432, torque control 434, and an adaptive gripping head 436. When activated, the motor 432 engages the gripping head 436 to firmly secure the cap, then applies rotational torque to loosen and remove the cap. In some cases, a sensor 438 can be implemented within the cap opening unit 430 to adjust the gripping pressure and prevent slippage or over-tightening. In some embodiments, the cell vessel operation station 410 is a mechanical manipulator.

The torque control may regulate the rotational force applied by the motor during cap opening or closing operations. The torque control may limit the maximum torque applied to caps or lids to prevent damage to cell culture vessels or to the caps or lids. The torque control may monitor torque levels during cap opening operations to detect when a cap or lid has been loosened from a cell culture vessel. The torque control may monitor torque levels during cap closing operations to detect when a cap or lid has been tightened to a desired level on a cell culture vessel. In some cases, the torque control may comprise a torque sensor, a current-limiting circuit, or a slip clutch mechanism that limits or monitors the rotational force transmitted from the motor to the adaptive gripping head.

The adaptive gripping head may be configured to engage with caps or lids of cell culture vessels of various sizes and configurations. The adaptive gripping head may comprise gripping elements such as fingers, jaws, or pads that contact and secure caps or lids during cap opening or closing operations. The gripping elements of the adaptive gripping head may be compliant or adjustable to conform to caps or lids of different diameters, shapes, or surface textures. In some cases, the adaptive gripping head may comprise multiple gripping elements arranged in a radial pattern around a central axis to engage caps or lids from multiple directions.

The adaptive gripping head may be configured to engage with caps or lids of cell culture vessels of various sizes and configurations. The adaptive gripping head may comprise gripping elements such as fingers, jaws, or pads that contact and secure caps or lids during cap opening or closing operations. The gripping elements of the adaptive gripping head may be compliant or adjustable to conform to caps or lids of different diameters, shapes, or surface textures. In some cases, the adaptive gripping head may comprise multiple gripping elements arranged in a radial pattern around a central axis to engage caps or lids from multiple directions.

When activated, the motor may engage the adaptive gripping head to firmly secure a cap or lid of a cell culture vessel. The adaptive gripping head may apply gripping force to the cap or lid to establish a secure grip that prevents slippage during rotation. The motor may then apply rotational torque through the adaptive gripping head to loosen and remove the cap or lid from the cell culture vessel. During cap closing operations, the motor may apply rotational torque in an opposite direction to tighten the cap or lid onto the cell culture vessel.

At least one cell vessel operation station may be positioned within the operation hood to receive cell vessels for performing cell culture operations. The cell vessel operation station may provide a platform for securing and manipulating cell vessels during liquid handling procedures and other routine cell culture operations. In some cases, the operation hood may comprise multiple cell vessel operation stations to enable parallel processing of multiple cell vessels.

When the cell vessel operation station is a mechanical manipulator, the cell vessel operation station may comprise an articulated structure with multiple joints and segments that enable positioning and orienting cell culture vessels with multiple degrees of freedom. The mechanical manipulator configuration of the cell vessel operation station may enable the cell vessel operation station to perform complex motions such as rotation, bending, or twisting to position cell culture vessels at desired orientations during cell culture operations.

The cell vessel operation station configured as a mechanical manipulator may comprise a base joint, multiple articulated segments connected by joints, and actuators that drive movement of the articulated segments. The base joint may be mounted to the floor of the operation hood or to a base anchor within the operation hood. The multiple articulated segments may extend from the base joint and may be connected by rotary joints, linear joints, or combinations thereof. The actuators may be rotary actuators or linear actuators that drive movement of the articulated segments about or along the joints.

In some cases, the cell vessel operation station configured as a mechanical manipulator may have four to seven degrees of freedom. The degrees of freedom may include rotational degrees of freedom about different axes and may include translational degrees of freedom along different axes. A cell vessel operation station having four degrees of freedom may provide positioning capability with limited orientation control. A cell vessel operation station having five or six degrees of freedom may provide positioning and orientation capability sufficient for many cell culture operations. A cell vessel operation station having seven degrees of freedom may provide redundant motion capability that enables the mechanical manipulator to reach desired positions and orientations while avoiding obstacles or optimizing joint configurations.

The cell vessel operation station configured as a mechanical manipulator may comprise an end-effector configured to hold and manipulate cell culture vessels. The end-effector may be a gripper, platform, or other holding mechanism that secures cell culture vessels during cell culture operations. In some cases, the end-effector may comprise a heating plate and mechanical springs similar to those described for the cell vessel operation station to maintain cell culture vessels at controlled temperatures and in secure positions during manipulation.

The cell vessel operation station configured as a mechanical manipulator may be controlled to operate in synchronization with the robotic gantry system within the operation hood. The synchronized operation may enable coordinated movements between the cell vessel operation station and the robotic gantry system during cell culture operations such as media exchange or reagent addition. In some cases, the cell vessel operation station configured as a mechanical manipulator may position and orient a cell culture vessel while the robotic gantry system positions liquid handling devices for aspirating or dispensing liquids.

The mechanical manipulator configuration of the cell vessel operation station may enable the cell vessel operation station to perform motions that facilitate routine cell culture operations. The cell vessel operation station configured as a mechanical manipulator may tilt cell culture vessels to collect liquids at a corner or edge of the cell culture vessel for aspiration. The cell vessel operation station configured as a mechanical manipulator may rotate cell culture vessels to distribute liquids across cell culture surfaces. The cell vessel operation station configured as a mechanical manipulator may position cell culture vessels at orientations that provide access for the motorized cap opening unit or for liquid handling devices carried by the robotic gantry system.

FIG. 5 illustrates a prospective view of one of the embodiments of the robotic gantry system structured to operate inside the operation hood and operate in coordination with the at least one cell vessel operation station to perform automated cell culture operations on cell vessels positioned at the cell vessel operation station. The robotic gantry system 500 is framed within the space of the operation hood 400, having an X-axis 502 designed for horizontal axis movement, a Y-axis 504 mounted on the X-axis bridge to move perpendicular across the workspace, and a Z-axis 506 mounted on the Y-axis carriages that moves vertically to pick, place, approach, retract, or apply tools. All three axes 502, 504, 506 combine to position any payload anywhere within a 3D-rectangular working envelope.

The robotic gantry system may be a 3-axis mechanical motion platform coupled with at least one liquid handling device. The 3-axis mechanical motion platform may provide controlled movement of payloads such as liquid handling devices within a three-dimensional rectangular working envelope defined by the workspace of the operation hood. The coupling of the robotic gantry system with at least one liquid handling device may enable automated liquid handling operations such as aspirating media from cell culture vessels, dispensing fresh media into cell culture vessels, or adding reagents during cell culture operations.

The robotic gantry system 500 is a 3-axis mechanical motion platform coupled with at least one liquid handling device 510. The gantry system 500 moves any payload, such as any liquid handling device 510 carrying or dispensing liquid, in three linear directions along the X-axis, Y-axis, and Z-axis. The robotic gantry system 500 further comprises mounting points for rails, motors, and cable tracks. Each axis may ride on a bearing system, such as linear guide rails with recirculating ball carriages.

The robotic gantry system 500 further comprises a drive mechanism for precision positioning with speed and load, which may be belt drive, ball-screw drive, rack-and-pinion, or linear motors. The robotic gantry system 500 further comprises a motor feedback component such as servo motors with absolute or incremental encoders for closed-loop control, consistent torque across speeds, and precise, synchronized motion across axes. The Z-axis 506 of the robotic gantry system 500 is designed to lift and lower a liquid exchange and dispensing device or other parts and further comprises a high-resolution ball-screw hinge, an integrated mechanical brake, and a lightweight carriage to reduce inertia. The robotic gantry system 500 carries modular tools and parts such as grippers 540 (mechanical, pneumatic, or vacuum), cameras for inspection, dispensing heads, and any other modular tools. The robotic gantry system 500 comprises a cable system, wherein the cable system has power cables, encoder lines, pneumatic tubing, and sensing wiring.

The robotic gantry system 500 is connected to a control system 560 having at least one industrial motion controller and one PC-based controller that are connected to and modulated by an AI-command center of the cell culture system 100. The liquid handling device 510 may be an electronic pipette or an automatic device that may be suitable for exchanging liquid for the cell culture unit. The robotic gantry system 500 may have multiple Z-axes to carry out synchronized liquid dispensing and liquid handling tasks for more than one cell vessel. The liquid handling device may be dispensing heads configured to dispense liquids such as culture media, reagents, or other solutions into cell culture vessels. Dispensing heads may comprise nozzles, tips, or orifices through which liquids are expelled during dispensing operations. Dispensing heads may be connected to the liquid storage and supply system through liquid tubing routed along the robotic gantry system. In some cases, dispensing heads may comprise multiple nozzles for dispensing liquids into multiple locations simultaneously. In some cases, dispensing heads may be configured with valves or pumps that control the volume and flow rate of dispensed liquids.

In some embodiments, the liquid handling device may be an electronic pipette. An electronic pipette may comprise a motorized piston mechanism that aspirates and dispenses controlled volumes of liquid. The electronic pipette may include a motor that drives the piston mechanism and electronic controls that regulate the volume of liquid aspirated or dispensed. The electronic pipette may be configured to accept disposable tips that contact liquids during aspiration and dispensing operations. In some cases, the electronic pipette may be configured with programmable volume settings that enable the electronic pipette to aspirate or dispense different volumes during different steps of a cell culture protocol. The electronic pipette may receive control signals from the control system of the cell incubation system to perform automated liquid handling operations.

In some other embodiments, the liquid handling device may be an automatic device suitable for exchanging liquid for a cell culture unit. An automatic device suitable for exchanging liquid may comprise pumps, valves, and tubing configured to transfer liquids between reservoirs and cell culture vessels. The automatic device may be connected to the liquid storage and supply system of the cell incubation system through liquid tubing. The automatic device may be configured to aspirate spent media from cell culture vessels and to dispense fresh media into cell culture vessels during media exchange operations. In some cases, the automatic device may comprise peristaltic pumps, syringe pumps, or other pump mechanisms that generate controlled flow of liquids through the automatic device. In some cases, the automatic device may comprise flow sensors or volume sensors that monitor the amount of liquid transferred during liquid exchange operations. The automatic device may receive control signals from the control system of the cell incubation system to perform automated liquid exchange operations as part of routine cell culture protocols.

Additionally, at least one programmable mechanical manipulator 570 designed to mimic the movement and dexterity of a human arm with 2-7 degrees of freedom may be implemented within the operation hood 400 to operate in synchronization with the robotic gantry system 500 and perform motions such as rotation, bending, or twisting and to position and orient its end-effector (tool, gripper, sensor, etc.) with precision. In some embodiments, the mechanical manipulator 570 is a robotic arm with a base joint, multiple articulated segments connected by joints, rotary or linear actuators that drive movement, a wrist assembly for up to 7 DOF arms to control complex orientation, and an end-effector, such as a gripper, tool, or sensor. In some embodiments, a plurality of mechanical manipulators 570 may be configured to perform additional tasks in cell culture operations in coordination with the robotic gantry system 500.

Referring now to FIG. 6a, the robotic cell vessel transportation system 600 according to an embodiment of the present disclosure is illustrated. The robotic cell vessel transportation system 600 has at least one robotic arm 610 with a gripper 612 to pick up and transport the cell vessels and move the cell vessels to any designated locations such as a receiving plate 614 from the incubation chamber 200 through the shared window 150 to a microscopy observation station, or the cell vessel operation station 410.

Referring now to FIG. 7, the cell incubation system may further comprise a liquid storage and supply system configured as a closed system for storing and delivering liquids used in cell culture operations. The liquid storage and supply system 900 according to an embodiment of the present disclosure is illustrated. The liquid storage and supply system 900 is a closed system having liquid storage units located within a refrigerator, at least one liquid reservoir 440 located within the operation hood 400, a liquid heating and cooling system 920 that may be located anywhere outside the refrigerator, and a pressurized liquid tubing system 940 connecting the liquid storage units 910 and the liquid reservoir 440. The liquid reservoir 440 has a container positioned to be accessible for the pipette within the operation hood 400. The container of the liquid reservoir 440 is connected to the liquid tubing system 940.

The liquid storage and supply system may comprise liquid storage units located within a refrigerator, at least one liquid reservoir located within the operation hood, a liquid heating and cooling system, and a pressurized liquid tubing system connecting the liquid storage units and the liquid reservoir.

The liquid storage units may be containers, bottles, bags, or other vessels configured to hold cell culture media, reagents, wash solutions, or other liquids used during cell culture operations. The liquid storage units may be located within a refrigerator to maintain the liquids at reduced temperatures that preserve stability and extend shelf life of temperature-sensitive cell culture media and reagents. The refrigerator may maintain the liquid storage units at temperatures below ambient temperature, such as temperatures in a range of approximately 2 degrees Celsius to approximately 8 degrees Celsius, to inhibit degradation of media components and to reduce microbial growth within the stored liquids. In some cases, the liquid storage units may comprise multiple containers holding different types of liquids such as growth media, differentiation media, wash buffers, enzyme solutions, or other reagents used in cell culture protocols.

The liquid storage and supply system may comprise at least one liquid reservoir located within the operation hood. The at least one liquid reservoir may serve as an intermediate storage location for liquids that have been transferred from the liquid storage units within the refrigerator to the operation hood for use during cell culture operations. The at least one liquid reservoir may hold liquids at volumes and temperatures appropriate for immediate use by liquid handling devices of the robotic gantry system during cell culture operations. In some cases, the at least one liquid reservoir may comprise multiple liquid reservoirs, with each liquid reservoir holding a different type of liquid for different cell culture operations.

The liquid storage and supply system may comprise a liquid heating and cooling system configured to regulate the temperature of liquids as the liquids are transferred from the liquid storage units to the at least one liquid reservoir or to liquid handling devices. The liquid heating and cooling system may be located anywhere outside the refrigerator. In some cases, the liquid heating and cooling system may be positioned along the pressurized liquid tubing system between the refrigerator and the operation hood. In some cases, the liquid heating and cooling system may be positioned within or adjacent to the operation hood. In some cases, the liquid heating and cooling system may be positioned at a location remote from both the refrigerator and the operation hood.

The liquid heating and cooling system may comprise heating elements configured to raise the temperature of liquids from refrigerated storage temperatures to temperatures suitable for cell culture operations. The heating elements may warm cell culture media to temperatures approximating physiological conditions before the media is dispensed into cell culture vessels. In some cases, the liquid heating and cooling system may comprise cooling elements configured to maintain liquids at reduced temperatures during transfer or to cool liquids that have been heated above target temperatures. The liquid heating and cooling system may comprise temperature sensors and control circuitry configured to regulate operation of the heating elements and cooling elements to maintain liquids at target temperatures during transfer and dispensing operations.

The liquid storage and supply system 900 may comprise a pressurized liquid tubing system connecting the liquid storage units and the liquid reservoir. The pressurized liquid tubing system may comprise tubing, fittings, valves, and associated components that form fluid pathways between the liquid storage units within the refrigerator and the at least one liquid reservoir within the operation hood. The pressurized liquid tubing system may be configured to transfer liquids from the liquid storage units to the at least one liquid reservoir under positive pressure. In some cases, the positive pressure may be generated by pressurized gas such as compressed air or inert gas applied to the liquid storage units or to portions of the pressurized liquid tubing system. In some cases, the positive pressure may be generated by pumps positioned along the pressurized liquid tubing system.

The pressurized liquid tubing system may maintain the liquid storage and supply system as a closed system that isolates the stored liquids from external environments during storage and transfer. The closed system configuration may reduce contamination risk by preventing exposure of cell culture media and reagents to airborne contaminants during transfer from the liquid storage units to the at least one liquid reservoir. The pressurized liquid tubing system may comprise sterile filters, check valves, or other components that maintain sterility of the fluid pathways within the liquid storage and supply system.

The liquid reservoir may have a container positioned to be accessible for a pipette within the operation hood. The container of the liquid reservoir may be positioned at a location within the operation hood that enables liquid handling devices carried by the robotic gantry system to access the container for aspirating liquids during cell culture operations. In some cases, the container may be positioned on a surface or platform within the operation hood at a height and location that enables the Z-axis of the robotic gantry system to lower a pipette or other liquid handling device into the container to aspirate liquids. The positioning of the container to be accessible for a pipette may enable automated aspiration of cell culture media, reagents, or other liquids from the liquid reservoir by the robotic gantry system without requiring manual intervention.

The container of the liquid reservoir may be connected to the pressurized liquid tubing system. The connection between the container and the pressurized liquid tubing system may enable liquids to be transferred from the liquid storage units within the refrigerator to the container of the liquid reservoir within the operation hood. In some cases, the connection may comprise tubing fittings, quick-connect couplings, or other fluid connectors that establish fluid communication between the container and the pressurized liquid tubing system. The connection may enable the container to be refilled with liquids from the liquid storage units as liquids are consumed during cell culture operations. In some cases, the container may be configured with level sensors that detect when the liquid level within the container falls below a threshold, and the control system of the cell incubation system may initiate transfer of additional liquid from the liquid storage units to the container through the pressurized liquid tubing system in response to signals from the level sensors.

The liquid storage and supply system configured as a closed system with the liquid storage units located within the refrigerator, the at least one liquid reservoir located within the operation hood, the liquid heating and cooling system, and the pressurized liquid tubing system may enable automated delivery of temperature-conditioned liquids to the operation hood for cell culture operations while maintaining sterility and reducing contamination risk throughout the liquid handling process.

The robotic gantry system may comprise a cable system having power cables, encoder lines, pneumatic tubing, and sensing wiring. The cable system may route electrical and pneumatic connections along the moving axes of the robotic gantry system to supply power, transmit signals, and deliver compressed air to components mounted on the X-axis, Y-axis, and Z-axis.

The power cables of the cable system may supply electrical power to servo motors, actuators, and other electrically powered components of the robotic gantry system. The power cables may carry voltage and current levels appropriate for driving the servo motors of each axis and for powering tools carried by the robotic gantry system such as electronic pipettes, grippers, or dispensing heads. In some cases, the power cables may include separate conductors for different voltage levels or for different components of the robotic gantry system.

The encoder lines of the cable system may transmit position feedback signals from encoders of the servo motors to the control system of the robotic gantry system. The encoder lines may carry digital or analog signals representing position, velocity, or other motion parameters measured by absolute or incremental encoders mounted on the servo motors. The encoder lines may be shielded or twisted-pair cables configured to reduce electromagnetic interference that could affect accuracy of position feedback signals.

The pneumatic tubing of the cable system may deliver compressed air to pneumatically actuated components of the robotic gantry system. The pneumatic tubing may supply compressed air to pneumatic grippers, pneumatic cylinders, or other pneumatic actuators carried by or integrated with the robotic gantry system. The pneumatic tubing may be flexible tubing configured to accommodate movement of the X-axis, Y-axis, and Z-axis throughout their respective travel ranges.

The sensing wiring of the cable system may transmit signals from sensors distributed throughout the robotic gantry system. The sensing wiring may carry signals from limit switches, proximity sensors, force sensors, temperature sensors, or other sensors that monitor operating conditions or positions of components of the robotic gantry system. In some cases, the sensing wiring may transmit signals from sensors mounted on tools carried by the robotic gantry system to provide feedback during cell culture operations.

The cable system may be routed through cable tracks mounted along the X-axis, Y-axis, and Z-axis of the robotic gantry system. The cable tracks may protect the power cables, encoder lines, pneumatic tubing, and sensing wiring from damage during motion of the robotic gantry system. The cable tracks may maintain organized routing of the cables, lines, tubing, and wiring throughout the travel range of each axis and may prevent tangling or binding of the cable system during operation.

The robotic gantry system may comprise a liquid tubing system connected to a liquid storage system. The liquid tubing system may route liquid media, reagents, or other solutions from the liquid storage system to liquid handling devices carried by the robotic gantry system. The liquid tubing system may comprise flexible tubing configured to accommodate movement of the robotic gantry system along the X-axis, Y-axis, and Z-axis while maintaining fluid connections between the liquid storage system and the liquid handling devices.

The liquid tubing system may be connected to the liquid storage system of the cell incubation system. The liquid storage system may comprise liquid storage units, liquid reservoirs, and associated components for storing and supplying liquids used in cell culture operations. The liquid tubing system may draw liquids from the liquid storage units or liquid reservoirs and may deliver the liquids to dispensing heads, electronic pipettes, or other liquid handling devices mounted on the robotic gantry system. In some cases, the liquid tubing system may also return aspirated liquids from the liquid handling devices to waste containers of the liquid storage system.

The liquid tubing system may be routed alongside the cable system through cable tracks of the robotic gantry system. In some cases, the liquid tubing system may be routed through separate tubing guides or channels configured to accommodate the flexibility and routing requirements of liquid tubing. The liquid tubing system may comprise tubing materials compatible with cell culture media and reagents to prevent contamination or degradation of liquids during storage and transfer.

The robotic gantry system may be connected to a control system comprising at least one industrial motion controller and one PC-based controller connected to an AI-command center. The control system may coordinate operation of the robotic gantry system with other components of the cell incubation system to perform automated cell culture operations.

The at least one industrial motion controller may generate control signals for the servo motors of the X-axis, Y-axis, and Z-axis of the robotic gantry system. The industrial motion controller may receive position feedback signals from encoders of the servo motors through the encoder lines of the cable system. The industrial motion controller may implement closed-loop control algorithms that compare commanded positions with measured positions and generate motor drive signals to achieve precise positioning of the robotic gantry system. In some cases, the industrial motion controller may coordinate synchronized motion across multiple axes of the robotic gantry system to execute complex motion trajectories during cell culture operations.

The temperature and humidity control system 700 provides conditioned air flow for the areas of cell culture incubation and operations within the incubation chamber 200 and the operation hood 400. The conditioned air flow supplied to the operation hood may reduce thermal shock to cell cultures when cell vessels are transferred from the dark incubation chamber to the operation hood for cell culture operations. In some cases, the temperature and humidity control system may supply conditioned air flow to the operation hood through air handling components positioned within or adjacent to the operation hood.

In some cases, the temperature and humidity control system may be configured to supply conditioned air flow with controlled gas composition. The controlled gas composition may include elevated carbon dioxide levels to maintain pH buffering of bicarbonate-based cell culture media within the dark incubation chamber. In some cases, the controlled gas composition may include reduced oxygen levels for hypoxic cell culture applications. The temperature and humidity control system may comprise gas supply connections, gas mixing components, and gas sensors configured to regulate the composition of conditioned air flow supplied to the dark incubation chamber and the operation hood.

In some cases, the cell incubation system may include the waste collection and storage system 800 has a solid waste container 810 and a liquid waste container 820 for collecting waste materials generated during cell culture operations. The waste collection and storage system may be configured to manage waste materials generated during cell culture operations performed within the operation hood. The waste collection and storage system may receive and contain waste materials produced during routine cell culture operations such as media exchange, cell passage, enzyme digestion, and other procedures that generate solid or liquid waste.

The waste collection and storage system may comprise a solid waste container configured to receive solid waste materials generated during cell culture operations. The solid waste container may be configured to contain disposable items used during cell culture operations such as pipette tips, disposable tubing, cell scrapers, or other consumable materials. In some cases, the solid waste container may be configured to receive cell culture vessels that are no longer needed or that have been contaminated during cell culture operations. The solid waste container may be positioned within or adjacent to the operation hood to enable the robotic gantry system or the programmable mechanical manipulator to deposit solid waste materials into the solid waste container during automated cell culture operations. In some cases, the solid waste container may comprise a lid or cover that opens to receive solid waste materials and closes to contain the solid waste materials within the solid waste container. In some cases, the solid waste container may be removable from the waste collection and storage system to enable disposal of accumulated solid waste materials and replacement with an empty solid waste container.

The waste collection and storage system may comprise a liquid waste container configured to receive liquid waste materials generated during cell culture operations. The liquid waste container may be configured to contain spent cell culture media, wash solutions, enzyme solutions, or other liquid materials aspirated from cell culture vessels during cell culture operations. The liquid waste container may be connected to liquid handling devices of the robotic gantry system through tubing that routes aspirated liquids from the liquid handling devices to the liquid waste container. In some cases, the liquid waste container may be connected to the cell vessel operation station to receive liquids drained or aspirated from cell culture vessels positioned at the cell vessel operation station.

The liquid waste container may comprise a vessel or reservoir with sufficient volume capacity to contain liquid waste materials generated during extended periods of automated cell culture operations. In some cases, the liquid waste container may comprise level sensors configured to detect when the liquid waste container is approaching full capacity. The level sensors may provide signals to the control system of the cell incubation system to alert operators when the liquid waste container is due for emptying or replacement. In some cases, the liquid waste container may be removable from the waste collection and storage system to enable disposal of accumulated liquid waste materials and replacement with an empty liquid waste container.

In some cases, the waste collection and storage system may comprise separate liquid waste containers for different types of liquid waste materials. In some cases, a first liquid waste container may be configured to receive spent cell culture media, and a second liquid waste container may be configured to receive hazardous liquid waste materials such as enzyme solutions or chemical reagents. The separation of different types of liquid waste materials into separate liquid waste containers may facilitate appropriate disposal procedures for different waste categories.

The waste collection and storage system may be positioned within the cell incubation system to enable access by the robotic gantry system, the programmable mechanical manipulator, and the robotic cell vessel transportation system during automated cell culture operations. The positioning of the solid waste container and the liquid waste container within the waste collection and storage system may enable automated deposition of waste materials without requiring manual intervention during cell culture operations. The waste collection and storage system may contribute to maintaining cleanliness within the operation hood by providing designated containers for waste materials that would otherwise accumulate within the workspace during extended automated cell culture operations.

The cell incubation system may be adapted for AI-assisted culture monitoring and protocol generation. The AI-assisted culture monitoring may enable intelligent analysis of cell growth patterns observed through the cell image monitor system. The AI-assisted protocol generation may enable automated decision-making for routine culture operations such as media exchange, cell passage, and enzyme digestion. In some cases, the AI-assisted culture monitoring and protocol generation may reduce operator variability and ensure consistent execution of culture protocols across large numbers of cell vessels.

The one PC-based controller may provide higher-level control functions for the robotic gantry system and for the cell incubation system. The PC-based controller may execute software programs that define cell culture protocols, motion sequences, and operational parameters for the robotic gantry system. The PC-based controller may communicate with the industrial motion controller to transmit motion commands and to receive status information regarding operation of the robotic gantry system. In some cases, the PC-based controller may provide a user interface for operators to configure, monitor, and control the cell incubation system.

The PC-based controller may be connected to an AI-command center of the cell incubation system. The AI-command center may provide AI-assisted culture monitoring and protocol generation capabilities for the cell incubation system. The connection between the PC-based controller and the AI-command center may enable the AI-command center to transmit commands to the robotic gantry system through the PC-based controller and the industrial motion controller. In some cases, the AI-command center may analyze data from the cell image monitor system and may generate automated decisions for routine cell culture operations that are executed by the robotic gantry system under control of the PC-based controller and the industrial motion controller.

The control system comprising the at least one industrial motion controller and the one PC-based controller connected to the AI-command center may enable automated execution of cell culture protocols with reduced manual intervention. The hierarchical control architecture with the AI-command center, the PC-based controller, and the industrial motion controller may provide intelligent decision-making at the AI-command center level, protocol management and user interface functions at the PC-based controller level, and precise motion control at the industrial motion controller level.

When the AI-assisted culture monitoring determines that a cell culture operation is warranted, or when a scheduled cell culture operation is due according to a cell culture protocol, the control system may initiate a sequence of coordinated actions involving the robotic cell vessel transportation system, the operation hood, and the robotic gantry system. The robotic cell vessel transportation system may retrieve a cell vessel from the dark incubation chamber by extending the robotic arm with the gripper to engage with the cell vessel on a shelf rack. In some cases, the shelf rack may rotate to position the cell vessel at an angular position accessible to the robotic arm. The robotic arm may transport the cell vessel through the shared window between the dark incubation chamber and the operation hood.

Within the operation hood, the robotic cell vessel transportation system may place the cell vessel at a cell vessel operation station. The cell vessel operation station may secure the cell vessel on the heating plate using the mechanical springs that provide a snug fit within the space formed by the heating plate and the peripheral guard wall. The heating plate may maintain the cell vessel at a controlled temperature during the cell culture operation. In some cases, the wrist assembly and rotary mechanism of the cell vessel operation station may position the cell vessel at a tilted orientation to facilitate liquid handling procedures.

The motorized cap opening unit may engage with the cap or lid of the cell vessel and may apply rotational torque to loosen and remove the cap or lid. The adaptive gripping head of the motorized cap opening unit may secure the cap or lid, and the motor may rotate the cap or lid to open the cell vessel. In some cases, the programmable mechanical manipulator may hold the removed cap or lid while the robotic gantry system performs liquid handling operations on the cell vessel.

The robotic gantry system may position liquid handling devices over the cell vessel at the cell vessel operation station. The X-axis, Y-axis, and Z-axis of the robotic gantry system may operate in coordination to move the liquid handling devices to positions above the cell vessel for aspirating spent media or dispensing fresh media. The liquid handling device, which may be an electronic pipette or an automatic liquid dispensing device, may aspirate spent media from the cell vessel. The aspirated spent media may be routed through the liquid tubing system to the liquid waste container of the waste collection and storage system.

The robotic gantry system may then position the liquid handling device over the liquid reservoir within the operation hood to aspirate fresh media. The liquid reservoir may contain media that has been transferred from the liquid storage units within the refrigerator through the pressurized liquid tubing system and that has been temperature-conditioned by the liquid heating and cooling system. The liquid handling device may aspirate fresh media from the liquid reservoir and may dispense the fresh media into the cell vessel at the cell vessel operation station. In some cases, the cell vessel operation station may tilt or rotate the cell vessel during dispensing to distribute the fresh media across the cell culture surface.

After the liquid handling operation has been completed, the motorized cap opening unit may replace the cap or lid on the cell vessel. The motor may apply rotational torque in the opposite direction to tighten the cap or lid onto the cell vessel. The torque control may regulate the tightening force to prevent over-tightening. In some cases, the sensor of the motorized cap opening unit may provide feedback to adjust gripping pressure during the cap closing operation.

The robotic cell vessel transportation system may retrieve the cell vessel from the cell vessel operation station and may transport the cell vessel back to the dark incubation chamber through the shared window. The robotic arm may place the cell vessel on a shelf rack of the multi-level shelving assembly for continued incubation. In some cases, the robotic cell vessel transportation system may transport the cell vessel to the microscopy observation station for imaging by the cell image monitor system before returning the cell vessel to the shelf rack.

The coordination between the components of the cell incubation system may enable parallel processing of multiple cell vessels. In some cases, the robotic cell vessel transportation system may transport a first cell vessel to the operation hood for cell culture operations while a second cell vessel remains at the microscopy observation station for imaging. In some cases, the robotic gantry system having multiple Z-axes may perform synchronized liquid dispensing operations on multiple cell vessels positioned at adjacent cell vessel operation stations. The parallel processing capability may increase throughput of the cell incubation system compared to sequential processing of individual cell vessels.

The integration of the components within the unified platform may reduce contamination risk by minimizing the frequency with which cell vessels are exposed to external environments. In conventional cell culture workflows, cell vessels may be removed from incubators, transported to separate workstations, and handled under biosafety cabinets before being returned to incubators. Each transfer between separate instruments may expose cell cultures to environmental fluctuations and potential contamination sources. In the cell incubation system, cell vessels may remain within the controlled zones of the dark incubation chamber and the operation hood throughout cell culture operations. The shared window between the dark incubation chamber and the operation hood may enable transfer of cell vessels between the two zones without exposing the cell vessels to external environments. The temperature and humidity control system may supply conditioned, filtered air flow to both the dark incubation chamber and the operation hood to maintain consistent environmental conditions throughout the cell incubation system.

The AI-assisted culture monitoring and protocol generation may enable intelligent analysis of cell growth patterns and automated decision-making for routine cell culture operations. The AI-command center may receive image data from the cell image monitor system and may analyze the image data to assess cell confluence, detect morphological changes, identify contamination events, or evaluate other parameters relevant to cell culture health and progress. Based on the analysis, the AI-command center may generate decisions regarding timing of media exchange, initiation of cell passage procedures, or other interventions. The AI-command center may transmit commands to the control system to execute the decided interventions using the robotic gantry system, the robotic cell vessel transportation system, and other automated components of the cell incubation system.

The AI-assisted protocol generation may enable the cell incubation system to adapt cell culture protocols based on observed cell behavior. In some cases, the AI-command center may adjust the timing of media exchanges based on observed consumption rates or pH changes detected through imaging. In some cases, the AI-command center may initiate cell passage procedures when cell confluence reaches a threshold level determined from image analysis. The adaptive protocol generation may reduce operator variability by applying consistent decision criteria across large numbers of cell vessels and may ensure that cell culture operations are performed at appropriate times based on actual cell culture conditions rather than fixed schedules.

The cell incubation system may be adapted for large-volume cell, tissue, and organoid culture operations. The multi-level shelving assembly with rotatable shelf racks may provide storage capacity for large numbers of cell vessels within the dark incubation chamber. The automated handling provided by the robotic cell vessel transportation system, the robotic gantry system, and the programmable mechanical manipulator may enable continuous, unattended culture operations that would be impractical with conventional manual methods. The liquid storage and supply system may provide sufficient volumes of cell culture media and reagents to support extended automated culture operations without requiring frequent manual replenishment.

The cell incubation system may address scalability limitations inherent in manual cell culture approaches. In conventional workflows, the number of cell vessels that can be maintained may be limited by the labor resources available for performing routine cell culture operations. The automated operation of the cell incubation system may enable a single operator to oversee culture operations for large numbers of cell vessels that would otherwise require multiple laboratory personnel. The reduced labor demand may lower operational costs and may enable research and production programs to scale cell culture operations to meet increased demand for cells, tissues, or organoids.

The cell incubation system may provide improved consistency in cell culture operations compared to manual approaches. The robotic gantry system may perform liquid handling operations with repeatable volumes, positions, and timing. The cell vessel operation station may position cell vessels at consistent orientations during liquid handling procedures. The temperature and humidity control system may maintain consistent environmental conditions throughout incubation and cell culture operations. The AI-assisted protocol generation may apply consistent decision criteria for initiating cell culture operations. The combination of these automated and controlled elements may reduce variability in cell culture outcomes that can arise from differences in manual technique, timing, or environmental conditions in conventional workflows.

The cell incubation system may enable continuous monitoring of cell cultures through the cell image monitor system without disturbing the controlled environment of the dark incubation chamber. In conventional workflows, microscopic observation may require removing cell vessels from incubators and transporting the cell vessels to separate microscopy stations. The removal and transport may expose cell cultures to environmental fluctuations and may interrupt incubation. The cell image monitor system of the cell incubation system may capture images of cell cultures within the controlled environment, enabling observation without the disruptions associated with conventional microscopy procedures. The continuous monitoring capability may enable early detection of morphological changes, contamination events, or other developments that could otherwise go unnoticed between scheduled manual inspections.

A number of implementations have been described. Nevertheless, it will be understood that various modifications may be made without departing from the spirit and scope of the disclosure. Accordingly, other implementations are within the scope of the following claims.

1. A multi-stage thermal energy storage ("mTES") unit comprising:
a concentric multi-layer shell, each shell made from any thermal insulation material;
a plurality of shell-shaped chambers defined by adjacent shells wherein the chambers are filled with a thermal storage material that can be solid, fluid material, or phase-changing material, wherein the shell-shaped chambers are filled with the thermal storage materials that determine a minimum thermal capacity of any one of the shell- shaped chambers represented by a temperature Ts;
a core-chamber defined by the shell that is nearest to the geometric center of the mTES wherein the core-chamber is filled with the thermal storage materials that determine a maximum thermal capacity of the core-chamber represented by a temperature Tc; and
an inlet and an outlet connected to each of the concentric multi-layer shells to allow a heat transfer material to travel through each chamber, including each of the plurality shell-shaped chambers and the core-chamber, independently wherein the heat transfer materials may be fluid materials such as gas or liquid.

2. The mTES unit, according to claim 1, wherein said thermal storage material is a plurality of solid bricks that stacks up, each solid brick has a geometric design and, when being stacked up, will form a plurality of fluid channels in between bricks and allow the heat transfer materials flow through the fluid channels and travel through the shell-shaped chambers and the core-chamber in a controlled speed between the chambers' inlet and outlet.

3. The thermal storage material in claim 2 wherein the individual solid brick's geometric design is patterned grooves on at least one of the surfaces of the brick.

4. The mTES unit in claim 1 further comprises: a plurality tubes embedded within the thermal storage materials to carry the heat transfer materials that flow through the thermal storage materials wherein the plurality tubes are converged at two points where one converging point is connected to the inlet of the shell, and the other converging point is connected to the outlet of the shell.

5. The mTES unit, according to claim 1, the concentric multi-layer shell that is furthest to the center of the unit is made from materials that is optimized to sustain pressure operation inside the TES unit.

6. The mTES unit, according to claim 1, the concentric mTES of each chamber is made from a thermal insulation material.

7. The mTES unit in, according to claim 1, the concentric multi-layer shell of each chamber has a minimum thickness of design to control heat from diffusing into adjacent chambers, wherein each shell's thickness may be calculated to be the same or different.

8. The mTES unit, according to claim 1, the size of each chamber is calculated to have space to house sufficient thermal storage materials that hold the desired amount of energy within the chamber's predetermined temperature ranges as different temperature stages.

9. The mTES unit, according to claim 1, wherein the outlet of the core chamber has an optional connection with at least one inlet of at least one shell-chamber and forms a direct heat exchange loop between the core-chamber and any of the shell chambers.

10. 10. A method for charging an mTES unit, comprising steps of:
having an mTES unit, wherein the mTES has a core-chamber filled with the thermal storage material, and the core-chamber is surrounded with concentric multi-layer heat insulation shells wherein adjacent shells forming a plurality of shell-shaped chambers and each shell-shaped chambers and core-chamber are filled with thermal storage materials, each shell has an independent inlet and outlet open to the space inside the shell-shaped chambers or core- chamber, said inlet and outlet are connected to a heat source and to allow the heat transfer materials travel through all the shell- chambers, and the core-chamber;
having one of the shell-shaped chambers or the core-chamber be a receiving chamber and receiving the heat transfer material from the heat source wherein the heat source provides the heat transfer material with a temperature higher than the receiving chamber, wherein the heat transfer material travel with a controlled pressure through the receiving chambers via the inlet that connects to the receiving chamber and allows heat exchange with the thermal storage materials within the receiving chamber;
releasing the heat transfer material from the receiving chambers through the outlet of the receiving chamber;
continuously receiving the heat transfer material from the heat source and releasing the heat transfer material after it travels through the receiving chambers until after said receiving chambers reach a targeted temperature; and
directing the heat transfer material to bypass the mTES unit and rejoin the flow from the heat source when the temperature at the outlet of the receiving chamber reaches a predetermined temperature range.

11. A method for charging an mTES unit according to claim 10,
wherein the heat source may be a plurality of mTES that are connected with each other in sequential or parallel via channels or valves, wherein when at least one shell shaped-chambers or core-chamber has a temperature within a first mTES is lower than the released heat transfer material from any chambers from a second mTES, the first mTES may receive the high temperature heat transfer material from the second mTES.

12. A method for charging an mTES unit according to claim 10,
wherein when the heat transfer material released from any one of the shell- shaped chambers or core-chamber has a temperature higher than the temperature of other chambers, the heat transfer with higher temperature may be rerouted back to the inlet of the chamber that has a lower temperature to continue the heat exchange with the heat storage material inside the same mTES unit;

13. A method for charging an mTES unit according to claim 10 wherein the heat source may be a power generator that can output heated transfer material such as hot steams or hot air from a power field, concentrated solar thermal heat, geothermal heat, electric heater, furnace, process waster heat.

14. A method for discharging an mTES unit, comprising steps of:
having at least one mTES unit to be connected to at least one thermal energy consumption device, and the mTES has a core- chamber filled with heat storage material, and the core-chamber is surrounded with a concentric multi-layer heat insulation shells wherein adjacent shells forming a plurality of shell-shaped chambers and each chamber are filled with thermal storage materials, each shell has an independent inlet and outlet open to the space inside the shell-shaped chamber or core-chamber and connects to a heat source to allow any heat transfer materials travel through the core-chamber and the shell-shaped chambers;
allowing the heat transfer material from at least one of the chambers from at least one mTES unit to be released to the thermal energy consumption device through the outlet of the chamber connected with the thermal energy consumption device, wherein the thermal energy consumption device exchange the thermal value with the heat transfer material and converts the thermal energy to another form of energy such as electricity and resulting the heat transfer material after the exchange with a lower thermal value; and
directing the heat transfer material with a lower thermal value after the thermal energy consumption device to a second thermal energy consumption device that may utilize the thermal value or directing the heat transfer material to rejoin the heat source or release the heat transfer material into the air.

15. A method for discharging an mTES unit, according to claim 14,
wherein the thermal energy consumption device can be a power generator that converts thermal energy into another form of energy, such as electricity.

16. A method for discharging an mTES, according to claim 14,
wherein the thermal energy consumption device can be a terminal device that directly radiates thermal energy into the environment.

17. A method for discharging an mTES, according to claim 14,
wherein the thermal energy consumption device can be another multi-stage thermal energy storage unit with a core chamber temperature lower than the temperature of the released heat transfer material from the discharging mTES.

18. A method of operating the thermal energy storage unit using the mTES unit, according to claim 10, the method comprising:
having an mTES unit, wherein the mTES has a core-chamber filled with the thermal storage material, having a core heat generation device resides inside the core-chamber, wherein the core heat generation device may be powered by electricity, magnetic field, microwave, radiation, laser, radio frequency, chemical reaction, or any physical force, and the core-chamber is surrounded with concentric multi-layer heat insulation shells wherein adjacent shells forming a plurality of shell-shaped chambers and each shell-shaped chambers and core-chamber are filled with thermal storage materials, each shell has an independent inlet and outlet open to the space inside the shell-shaped chambers or core-chamber, said inlet and outlet are connected to a heat source and to allow the heat transfer materials travel through all the shell-chambers, and the core-chamber;
having one of the shell-shaped chambers or the core-chamber be a receiving chamber and receiving the heat transfer material from the heat source. wherein the core heat generation device provides the heat transfer material with a temperature higher than the receiving chamber, wherein the heat transfer material travel with a controlled pressure through the receiving chambers via the inlet that connects to the receiving chamber and allows heat exchange with the thermal storage materials within the receiving chamber;
releasing the heat transfer material from the receiving chambers through the outlet of the receiving chamber;
continuously receiving the heat transfer material from the heat source and releasing the heat transfer material after it travels through the receiving chambers until after said receiving chambers reach a targeted temperature; and
directing the heat transfer material to bypass the mTES unit and rejoin the flow from the heat source when the temperature at the outlet of the receiving chamber reaches a predetermined temperature range.
